(19) United States
(12) Patent Application Publication
(10) Pub. No.: US 2023/0233135 A1
(43) Pub. Date: Jul. 27, 2023

(54) SYMPATHETIC NERVOUS SYSTEM RESPONSE TO STIMULATION OF CAROTID BODIES FOR PATIENT STRATIFICATION IN RENAL DENERVATION

(71) Applicant: KONINKLIJKE PHILIPS N.V., Eindhoven (NL)

(72) Inventors: James David CEZO, SAN DIEGO, CA (US); Becky INDERBITZEN, SAN DIEGO, CA (US)

(21) Appl. No.: 18/098,880

(22) Filed: Jan. 19, 2023

Related U.S. Application Data
(60) Provisional application No. 63/302,451, filed on Jan. 24, 2022.

(51) Int. Cl. A61B 5/00; A61B 5/0215; A61B 5/242

(52) U.S. Cl. CPC A61B 5/4035; A61B 5/02158; A61B 5/242; A61B 2562/0247; A61B 2562/0261

(57) ABSTRACT

A system includes a processor circuit in communication with an anatomical measurement device. The anatomical measurement device receives a metric associated with a sympathetic response of a patient. The sympathetic nervous system of the patient is then stimulated. The anatomical measurement device then receives another metric associated with a sympathetic response of the patient while the sympathetic nervous system is stimulated. The processor circuit then provides an output based on the comparison.

### CROSS-REFERENCE TO RELATED APPLICATIONS

This application claims priority to and the benefit of U.S. Provisional Application No. 63/302,451, filed Jan. 24, 2022, which is incorporated by reference herein in its entirety.

### TECHNICAL FIELD

The present disclosure relates generally to renal denervation. In particular, a patient's sympathetic nervous system is monitored during stimulation of the renal nerves by stimulating a carotid body within a carotid artery to stratify patients based on their likelihood to respond to a renal denervation procedure.

### BACKGROUND

Physicians use many different medical diagnostic systems and tools to monitor a patient's health and diagnose medical conditions. In the field of assessing and treating hypertension in patients, various systems and devices are used to monitor a patient's condition and perform treatment procedures. One treatment procedure used to address hypertension of a patient is renal denervation. Renal denervation involves ablating or otherwise disabling the nerves of the renal artery. Because the renal nerves cause the renal artery to expand or contract in response to various stimuli, the renal nerves may be a cause of unnecessary high blood pressure in a patient. By disabling these nerves, blood pressure may be decreased.

However, renal denervation is not an effective treatment in all patients or at all locations within the renal vasculature of a patient. It is often difficult for a physician to determine whether a renal denervation will effectively address hypertension for a patient as results of renal denervation are highly patient-specific. As a result, a physician may perform a renal denervation procedure without success. This may be because the patient was not a patient which would respond positively to a renal denervation procedure or because the renal denervation procedure was performed in an incorrect region of the renal vasculature. Performing a renal denervation procedure with little to no effect on the patient unnecessarily subjects a patient to a traumatic and time-consuming procedure and wastes costly resources.

### SUMMARY

Embodiments of the present disclosure are systems, devices, and methods for stratifying patients for renal denervation based on monitoring sympathetic nervous response to carotid body stimulation. Aspects of the disclosure advantageously assist physicians in determining whether a patient would be an appropriate candidate for a renal denervation procedure and whether a renal denervation procedure performed previously was effective.

In some aspects, a sympathetic nervous system of a patient may be monitored while under stimulation and while at rest. The sympathetic nervous system may be monitored by an intraluminal device including pressure sensors, flow sensors, strain sensors, or electrodes, or by an extraluminal device including strain sensors or electrodes. The monitoring device may acquire metrics related to the sympathetic nervous system of the patient while the sympathetic nervous system is under stimulation by stimulating the carotid bodies of the patient and while the sympathetic nervous system is not stimulated or at rest. These metrics may be displayed to the user via the screen display as numerical values, or any suitable type of visual or graphical representation. The carotid bodies of the patient may be stimulated by applying external pressure to a region of the patient's neck corresponding to the carotid bodies, by an external patch with electrodes, or with an endovascular device. A processor circuit may receive the metrics acquired under stimulation and at rest. The processor circuit may compare the metrics. If a renal denervation procedure has not been performed and if a difference in the metrics exceeds a threshold, the processor circuit determines that the patient is a good candidate for renal denervation. If a renal denervation procedure was already performed, the processor circuit may determine that it was not successful and recommend additional treatment. If a renal denervation procedure has not been performed and if the difference in the metrics do not exceed a threshold, the processor circuit determines that the patient is not a good candidate for renal denervation. If a renal denervation procedure was already performed, the processor circuit may determine that it was successful.

In an exemplary aspect, a system is provided. The system comprises a processor circuit configured for communication with an anatomical measurement device, wherein the processor circuit is configured to: receive, from the anatomical measurement device, a first metric associated with a first sympathetic response of the patient while a sympathetic nervous system of the patient is not under stimulation; generate a visual representation of the first metric; receive, from the anatomical measurement device, a second metric associated with a second sympathetic response of the patient while the sympathetic nervous system of the patient is under the stimulation, wherein the stimulation of the sympathetic nervous system comprises stimulation of a carotid body of the patient; generate a visual representation of the second metric; and output a screen display to a display in communication with the processor circuit, wherein the screen display comprises the visual representation of the first metric and the visual representation of the second metric.

In one aspect, the processor circuit is configured to perform a comparison of the first metric and the second metric; and determine, based on the comparison, a likelihood of success of a future renal denervation procedure for the patient, wherein the screen display comprises a visual representation based on the likelihood of success. In one aspect, the processor circuit is configured to perform a comparison of the first metric and the second metric; and determine, based on the comparison, a degree of success a completed renal denervation procedure for the patient, wherein the screen display comprises a visual representation based on the degree of success. In one aspect, the anatomical measurement device comprises an endovascular catheter or guidewire configured to be positioned within a blood vessel of a patient. In one aspect, the blood vessel comprises a renal artery of the patient. In one aspect, the endovascular catheter or guidewire comprises one or more pressure sensors and one or more flow sensors, and wherein the processor circuit is configured to determine a fluid resistance measurement based on data received from the one or more pressure sensors and the one or more flow sensors. In one aspect, the endovascular catheter or guidewire comprises a strain sensor. In one aspect, the endovascular catheter or guidewire comprises one or more electrodes configured to measure an electrical field. In one aspect, the anatomical measurement device is configured to be positioned outside of the patient and in contact the patient's skin. In one aspect, the anatomical measurement device comprises a strain sensor. In one aspect, the anatomical measurement device includes one or more electrodes configured to measure an electrical field. In one aspect, the processor circuit is configured for communication with a stimulation device, and wherein the processor circuit is configured to control the stimulation device to provide the stimulation of the carotid body. In one aspect, the stimulation device comprising an endovascular catheter or guidewire configured to be positioned within a carotid artery of the patient. In one aspect, the stimulation device is configured to be positioned outside of the patient. In one aspect, the stimulation device comprises one or more electrodes configured to provide the stimulation of the carotid body. In one aspect, the stimulation of the carotid body comprises application of external pressure to a neck of the patient at a region comprising the carotid body.

In an exemplary aspect, a method is provided. The method includes receiving, with a processor circuit, first metric associated with a first sympathetic response of the patient from an anatomical measurement device in communication with the processor circuit, wherein the first metric is obtained by the anatomical measurement device while a sympathetic nervous system of the patient is not under stimulation; generating, with the processor circuit, a visual representation of the first metric; receiving, with the processor circuit, a second metric associated with a second sympathetic response of the patient from the anatomical measurement device, wherein the second metric is obtained by the anatomical measurement device while the sympathetic nervous system of the patient is under stimulation, wherein the stimulation of the sympathetic nervous system comprises stimulation of a carotid body of the patient; generating, with the processor circuit, a visual representation of the second metric; and outputting, with the processor circuit, a screen display to a display in communication with the processor circuit, wherein the screen display comprises the visual representation of the first metric and the visual representation of the second metric.

In an exemplary aspect, a system is provided. The system includes an anatomical measurement device; and a processor circuit configured for communication with the anatomical measurement device and a display, wherein the processor circuit is configured to: receive, from the anatomical measurement device, a first metric associated with a first sympathetic nervous system response of a patient while a carotid body of the patient is not under stimulation; receive, from the anatomical measurement device, a second metric associated with a second sympathetic nervous system response of a patient while the carotid body is under the stimulation, the stimulation of the carotid body causing a change from the first sympathetic nervous system response to the second sympathetic nervous system response; generate a screen display comprising a visual representation of the first metric and a visual representation of the second metric; and output the screen display to the display.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a flow diagram of a method 100 of patient stratification in renal denervation and assessment of success of renal denervation based on sympathetic nervous system response to simulation of carotid bodies, according to aspects of the present disclosure.

FIG. 2 is a schematic diagram of a data acquisition and carotid bodies stimulation system, according to aspects of the present disclosure.

FIG. 3 is a diagrammatic view of an endovascular device positioned within a renal anatomy, according to aspects of the present disclosure.

FIG. 4 is a diagrammatic view of an endovascular device within a renal artery and a carotid artery of a patient anatomy, according to aspects of the present disclosure.

FIG. 5 is a diagrammatic view of an endovascular device within a renal artery and an endovascular device within a carotid artery, according to aspects of the present disclosure.

FIG. 6 is a diagrammatic view of an endovascular device positioned within a renal anatomy and a carotid artery of a patient anatomy, according to aspects of the present disclosure.

FIG. 7 is a diagrammatic view of an endovascular device positioned within a renal anatomy, according to aspects of the present disclosure.

FIG. 8 is a diagrammatic view of an endovascular device positioned within a renal anatomy, according to aspects of the present disclosure.

FIG. 9 is a diagrammatic view of an endovascular device positioned within a branch of the renal anatomy, according to aspects of the present disclosure.

FIG. 10 is a diagrammatic view of an external device, according to aspects of the present disclosure.

FIG. 11 is a diagrammatic view of carotid arteries of a patient anatomy, according to aspects of the present disclosure.

FIG. 12 is a schematic diagram of a processor circuit, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

Aspects of the present disclosure may include various principles described in U.S. patent application Ser. No. 18/086,511, filed Dec. 21, 2022.

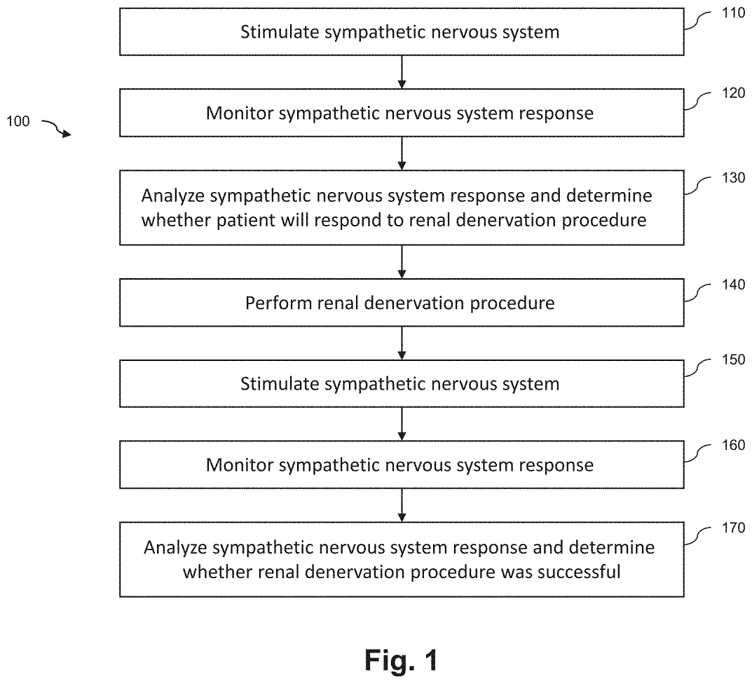

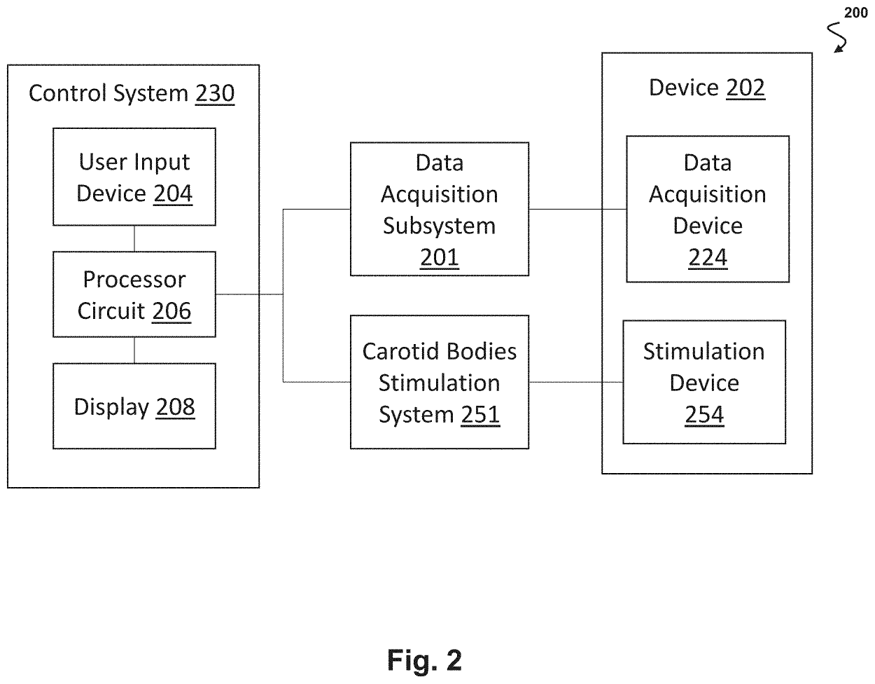

FIG. 1 is a flow diagram of a method 100 of patient stratification in renal denervation and assessment of success of renal denervation based on sympathetic nervous system response to simulation of carotid bodies, according to aspects of the present disclosure. The method 100 may describe an automatic segmentation of a vessel to detect segments of interest using co-registration of invasive physiology and x-ray images. As illustrated, the method 100 includes a number of enumerated steps, but embodiments of the method 100 may include additional steps before, after, or in between the enumerated steps. In some embodiments, one or more of the enumerated steps may be omitted, performed in a different order, or performed concurrently. The steps of the method 100 can be carried out by any suitable component within a diagnostic system and all steps need not be carried out by the same component. In some embodiments, one or more steps of the method 100 can be performed by, or at the direction of, a processor circuit of the diagnostic system 100, including, e.g., the processor 260 (FIG. 2) or any other component.

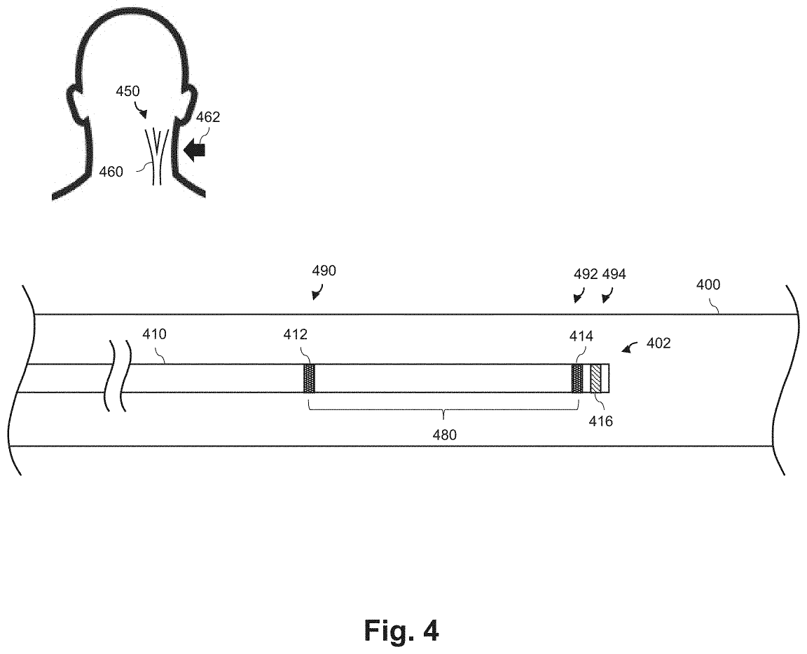

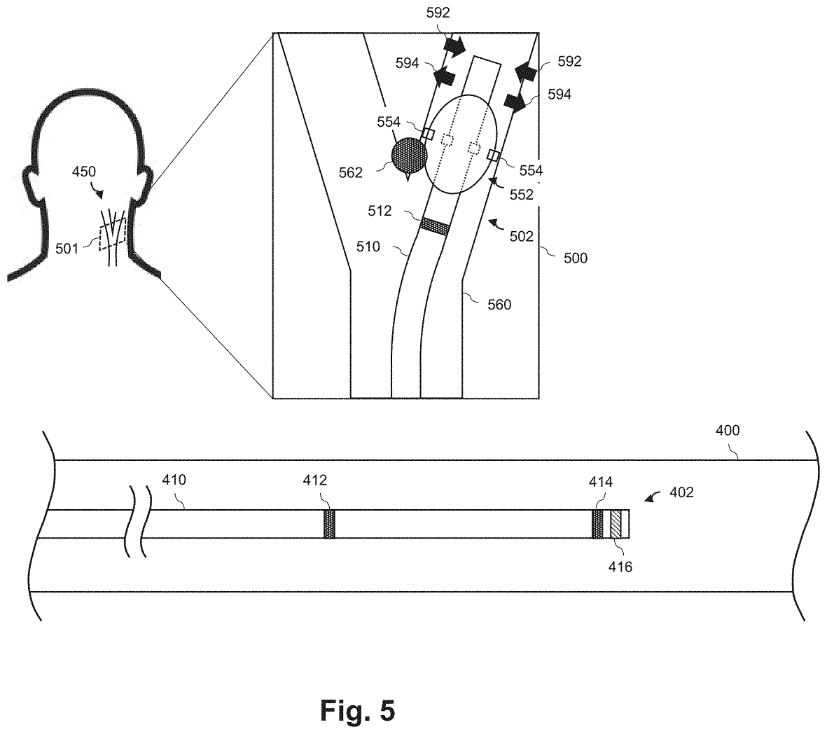

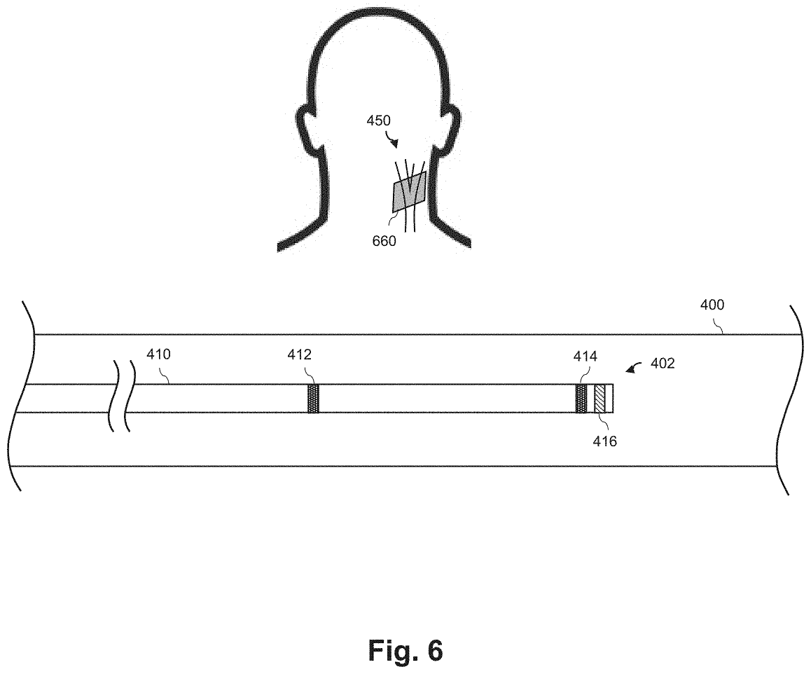

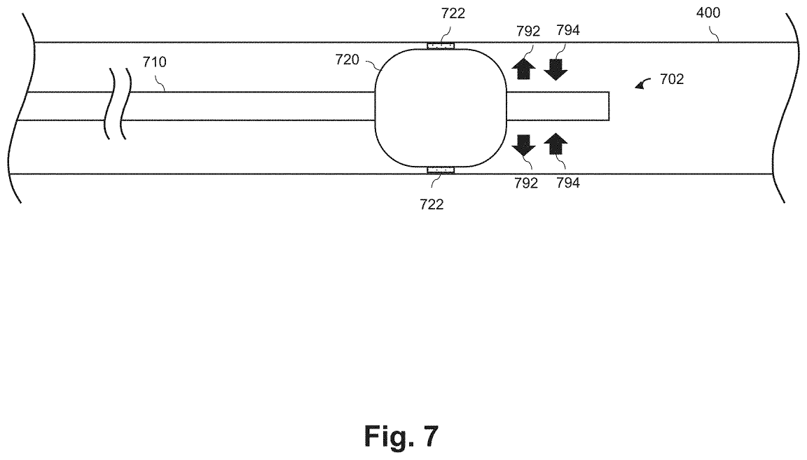

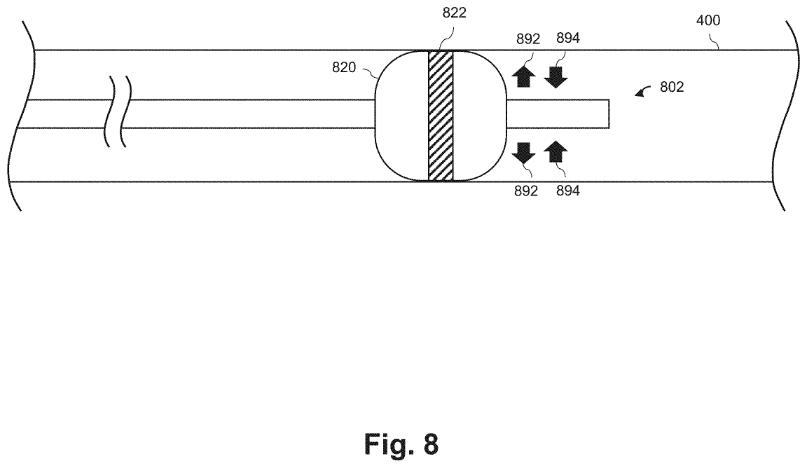

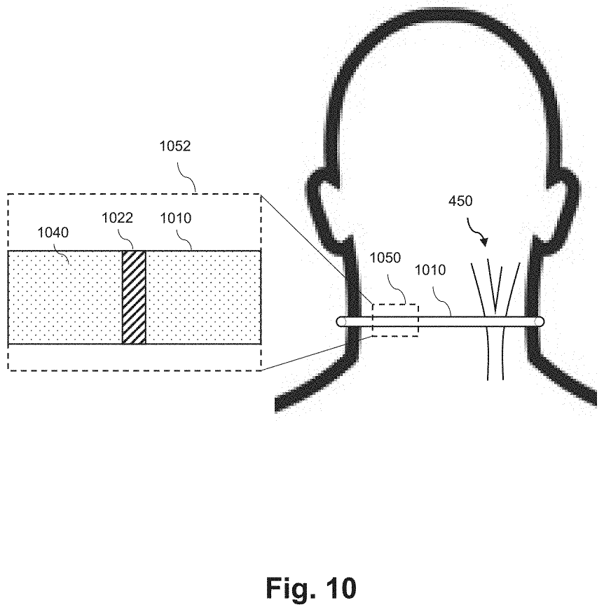

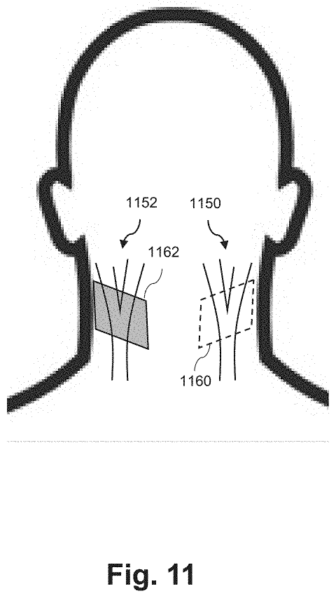

At step 110, the method 100 includes stimulating the sympathetic nervous system. The sympathetic nervous system may be stimulated in a variety of ways, as will be described in more detail with reference to the following figures. For example, as described with reference to FIG. 4, the sympathetic nervous system may be stimulated by applying pressure to the carotid bodies of the patient. This pressure may be applied in any suitable way. For example, a physician or technician, such as a user of the system 100, may apply pressure to either of the regions 1150 or 1152 (shown in FIG. 11) by pressing their hand or fingers at those regions. In some aspects, the pressure may be applied by an automated mechanism applied to the regions 1150 or 1152. In some aspects, the pressure may be applied via a wrap or other flexible elongate member wrapped around the neck of the patient. With reference to FIG. 5, the sympathetic nervous system may be stimulated by an endovascular device positioned within the renal artery of the patient. With reference to FIGS. 6 and 11, the sympathetic nervous system may be stimulated by an external patch positioned over the carotid bodies of the patient. With reference to FIGS. 7 and 8, the sympathetic nervous system may be stimulated by an endovascular device positioned within a renal artery of the patient. With reference to FIG. 10, the sympathetic nervous system may be stimulated by an external device.

At step 120, the method 100 includes monitoring the sympathetic nervous system for a response to the stimulation. The sympathetic nervous system response may be monitored in a variety of ways, including any of those described herein. For example, with reference to FIGS. 4-9, the sympathetic nervous system response may be monitored by an endovascular device positioned within a renal artery of a patient. In some aspects, the endovascular device may be referred to as an anatomical measurement device, a physiological measurement device, or any other suitable term. With reference to FIG. 10, the sympathetic nervous system response may be monitored by a strain sensor positioned against a patient's neck. With reference to FIG. 11, the sympathetic nervous system response may be monitored by electrodes of an external patch.

At step 130, the method 100 includes analyzing the sympathetic nervous system response and determining whether the patient will respond to a renal denervation procedure. In some aspects, analyzing the sympathetic nervous system response may include comparing metrics related to the sympathetic nervous system collected while the sympathetic nervous system was stimulated with metrics collected while the sympathetic nervous system was not stimulated. The metrics related to the sympathetic nervous system may include any of those described with reference to step 120 described above, or any other metrics described herein. For example, metrics may include a strain metric, mean arterial blood pressure, heart rate, blood flow, vascular impedance or conductance, or any other suitable metrics. For example, the system may acquire any of these metrics as a first metric while the sympathetic nervous system is not under stimulation. Then, the system may acquire a second metric of the same type as the first metric while the sympathetic nervous system is under stimulation. In that regard, step 130 of the method 100 may include comparing the first metric and the second metric. In that regard, the comparison may be a numerical value of a difference or a percentage difference. In some aspects, metrics obtained by a stimulation device or an anatomical measurement device may include blood pressure, blood flow, voltage measurements, strain measurements, or any other suitable measurements, values, or metrics. In some aspects, when a difference between metrics collected under stimulation and metrics collected while not under stimulation is observed, this may indicate that a patient will respond to a renal denervation procedure. In some aspects, this difference between metrics under stimulation and metrics not under stimulation (sometimes referred to as metrics at rest) may be compared to a threshold. For example, if this difference between metrics exceeds a predetermined threshold, a processor circuit of the system 200 may determine that the patient is likely to respond to a renal denervation procedure. In some aspects, when the sympathetic nervous system is stimulated, it may be referred to as undergoing stimulation, experiencing stimulation. In that regard, a stimulation device may provide stimulation.

In some aspects, the likelihood of success of a future renal denervation procedure may be calculated or provided in any suitable way. For example, if the future renal denervation procedure is determined to be likely, it may be provided as a binary or binary indication, such as the terms “yes”, “no”, “good candidate”, “bad candidate”, “recommended” and “not recommended” or any similar terms. In some aspects, if a future renal denervation procedure is likely, it may be provided as a value along a scale, or a term referring to a scale, such as “good”, “medium”, or “bad”, or “low”, “medium”, or “high”, referring to the degree of responsiveness of the sympathetic nervous system. In some aspects, the likelihood of success may be calculated and/or displayed as a value of a continuous numerical scale, such as a range of 1 to 10, 1 to 100, or any other suitable range.

In some aspects, step 130 of the method 100 may additionally include displaying any of the metrics previously described. For example, the system may output, to the display, the first metric obtained while the system is not under stimulation. The system may also or alternatively output, to the display, the second metric obtained while the system is under stimulation. In that regard, either of the first metric obtained while the sympathetic nervous system is not under stimulation or the second metric obtained while the sympathetic nervous system is under stimulation may be displayed as a graphical representation or a visual representation, including a numerical value, graph, chart, plot of values, or symbols. In some aspects, the graphical representation of the first metric and the graphical representation of the second metric may be simultaneously provided on a single screen display or on separate screen displays. In some aspects, the graphical representation of the first metric and the graphical representation of the second metric may be displayed individually at different times. In some examples, the graphical representation of the first metric and/or the graphical representation of the second metric may be displayed in response to a user input selecting the first metric or the second metric for display. In that regard, the graphical representation of the first metric and graphical representation of the second metric in response to the processor circuit receiving the first metric and/or the second metric and/or generating the graphical representations of either of the first metric and/or the second metric. In some aspects, the graphical representation of the first metric could be provided on the screen display first (i.e., only the graphical representation of the first metric without a display of the graphical representation of second metric) before the second metric is received or the graphical representation of the second metric is generated. After the second metric is received or the graphical representation of the second metric is generated, then the screen display may be updated or changed to additionally include the graphical representation of the second metric so that both are provided on the screen display simultaneously. In some aspects, the comparison of the first metric and the second metric may be output to the display as a visual or graphical representation on a graph, chart, plot of values, or any other suitable display. In some aspects, the comparison of the first metric and the second metric may highlight a difference between the first metric and the second metric.

At step 140, the method 100 includes performing a renal denervation procedure. A renal denervation procedure may include any procedure in which nerves surrounding the renal artery of a patient are disabled. For example, in some procedures, an endovascular device is positioned within the renal artery of the patient. With the device within the renal artery, electrodes of the device may ablate nerves surrounding the renal artery. This ablation procedure may be performed at various locations along either or both renal arteries of the patient. After a renal denervation procedure, the steps 110 through 130 may be performed again to determine if the renal denervation procedure was successful. In this way, steps 150 through 170 of the method 100 may be substantially similar to the steps 110 through 130 described above.

At step 150, the method 100 includes stimulating the sympathetic nervous system. Stimulating the sympathetic nervous system at step 150 may performed in a similar way as stimulating the sympathetic nervous system at step 110 to acquire controlled and comparable results. For example, if sympathetic nervous system was stimulated at step 110 by external electrodes at a given amplitude for a given amount of time, at step 150, the same procedure may be performed with the same electrodes, for the same amplitude, and for the same amount of time. In this way, any changes to the sympathetic response after the completion of a renal denervation procedure may be most accurately attributed to the renal denervation procedure.

At step 160, the method 100 includes monitoring the sympathetic nervous system for a response to the stimulation. Like the step 150 and step 110, the same method of monitoring may be used at step 160 as was used at step 120 to ensure accurate attribution of sympathetic nervous system response changes to the renal denervation procedure.

At step 170, the method 100 includes analyzing the sympathetic nervous system response and determining whether the renal denervation procedure was successful. In some aspects, analyzing the sympathetic nervous system response at step 170 may include comparing metrics related to the sympathetic nervous system collected while the sympathetic nervous system was stimulated (e.g., at step 150) with metrics collected while the sympathetic nervous system was not stimulated. After a renal denervation procedure has been performed, a difference between metrics collected under stimulation and metrics at rest this may indicate that the renal denervation procedure was not successful. This difference in response to stimulation may be due in part by the renal nerves still responding to the stimulation, meaning that they were not sufficiently disables. In some aspects, when there is little to no difference between metrics under stimulation and metrics at rest, it may indicate that the renal denervation procedure was successful. As described with reference to step 130, the comparison of metrics under stimulation and metrics at rest may include comparing these metrics to a threshold. In addition, differences between metrics collected at step 170 may be compared to differences between metrics collected at step 130. Any difference between these two differences in metrics may be compared to a predetermined threshold as well to determine whether a renal denervation procedure was successful.

In some aspects, any of the metrics described herein may be displayed to a user. For example, metrics obtained while the sympathetic nervous system is under stimulation may be displayed along with metrics obtained while the sympathetic nervous system is not under stimulation.

In some aspects, step 170 of the method 100 may additionally include displaying any of the metrics previously described. For example, the system may output, to the display, a metric obtained while the system is not under stimulation (e.g., an at-rest metric) after the renal denervation procedure was performed (see step 140). The system may also or alternatively output, to the display, an additional metric obtained while the system is under stimulation (e.g., an under-stimulation metric) and after the renal denervation procedure was performed. In that regard, either of the metric obtained while the sympathetic nervous system is not under stimulation or the metric obtained while the sympathetic nervous system is under stimulation may be displayed as a graphical representation or a visual representation, including a numerical value, graph, chart, plot of values, or symbols. In some aspects, the graphical representation of the at-rest metric and the graphical representation of the under-stimulation metric may be simultaneously provided on a single screen display or on separate screen displays. In some aspects, the graphical representation of the at-rest metric and the graphical representation of the under-stimulation metric may be displayed individually at different times. In some examples, the graphical representation of the at-rest metric and/or the graphical representation of the under-stimulation metric may be displayed in response to a user input selecting the at-rest metric or the under-stimulation metric for display. In that regard, the graphical representation of the at-rest metric and graphical representation of the under-stimulation metric in response to the processor circuit receiving the at-rest metric and/or the under-stimulation metric and/or generating the graphical representations of either of the at-rest metric and/or the under-stimulation metric. In some aspects, the graphical representation of the at-rest metric could be provided on the screen display first (i.e., only the graphical representation of the at-rest metric without a display of the graphical representation of under-stimulation metric) before the under-stimulation metric is received or the graphical representation of the under-stimulation metric is generated. After the under-stimulation metric is received or the graphical representation of the under-stimulation metric is generated, then the screen display may be updated or changed to additionally include the graphical representation of the under-stimulation metric so that both are provided on the screen display simultaneously. In some aspects, the comparison of the at-rest metric and the under-stimulation metric may be output to the display as a visual or graphical representation on a graph, chart, plot of values, or any other suitable display. In some aspects, the comparison of the at-rest metric and the under-stimulation metric may highlight a difference between the at-rest metric and the under-stimulation metric.

Aspects of the steps of the method 100 will be described with more detail throughout the description given hereafter. In some aspects, any of the systems, devices, sensors, methods, principles, or any teachings of the present invention may be substantially similar to the teachings of U.S. Provisional Application No. 63/300,536, filed Jan. 18, 2022, which is incorporated by reference herein in its entirety.

FIG. 2 is a schematic diagram of a data acquisition and carotid bodies stimulation system 200, according to aspects of the present disclosure. In some embodiments, and as shown in FIG. 2, the system 200 may include a control system 230, one or more subsystems, and one or more devices, such as the device 202.

The system 200 shown in FIG. 2 may advantageously assist a physician in assessing causes of hypertension in some patients and may assist a physician in determining whether a renal denervation procedure will likely be successful for a particular patient and/or whether a renal denervation procedure already performed was successful. In addition, the system 200 shown in FIG. 2 may be configured to identify whether a patient is likely to respond positively to a renal denervation procedure. For example, the system 200 may be configured to stimulate the sympathetic nervous system of the patient and measure a response to the stimulation. In some examples, the system 200 may stimulate the sympathetic nervous system by stimulating the carotid bodies of a patient. By analyzing the response of the patient to the stimulation of the carotid bodies, and by extension, sympathetic nervous system, the system 200 may be able to determine, based on the physiological response of the patient to stimulation, whether a patient's hypertension may be remedied or aided through a renal denervation procedure. The system 200 may also be able to quantify the effect of a previous renal denervation procedure on the response of the patient and therefore predict whether a previous renal denervation procedure was successful in remedying hypertension within the patient.

The control system 230 may be configured to generate various commands to control subsystems, such as the data acquisition subsystem 201 and/or the carotid bodies stimulation subsystem 251. The control system 230 may be additionally configured to generate commands to control various devices. For example, the control system 230 may be configured to generate commands to control the device 202. In some embodiments, the control system 230 may be configured to generate command signals to control one or more devices, such as the data acquisition device 224. The data acquisition device 224 may include various sensors, such as flow sensors, flow velocity sensors, pressure sensors, electrodes, strain sensors, or any other measurement devices. In addition, the control system 230 may be configured to generate command signals to control one or more devices, such as the stimulation device 254 shown in FIG. 2.

The control system 230 may be any suitable device or system. For example, the control system 230 may include a user input device 204, a processor circuit 206, and/or a display 208. The control system 230 may include additional devices, components, or elements. In some embodiments, the control system 230 may be a computer, such as a laptop, a tablet device, or any other suitable computational device. In some embodiments, the control system 230 may include additional elements related to communication between the control system 230, or the processor circuit 206 of the control system 230, and other systems, subsystems, or devices. For example, the control system 230 may include an interface module. In some examples, the control system 230 may include a patient interface module (PIM).

In some embodiments, the control system 230 may additionally be configured to receive various data from other systems, subsystems, or devices. For example, the control system may be configured to receive data related to blood flow, the velocity of blood within a vessel of a patient, pressure data, voltage measurements from an electrode, resistance and/or pressure measurements from a strain sensor, or any other type of data.

The user input device 204 may be any suitable device. For example, the user input device 204 may be configured to receive a user input via one or more buttons or mouse clicks. The user input device 204 may additionally be configured to receive a user input via any other method. For example, the user input device 204 may receive a user input via a touch on a touch screen, an auditory input such as speech or other sounds. In some embodiments, the user input device 204 may be a keyboard, a mouse, a touch screen, one or more buttons, a microphone, or any other suitable device configured to receive inputs from a user.

The processor circuit 206 may be configured to generate, receive, and or process any various data. For example, the processor circuit 206 may be in communication with the memory storage system of the control system 230. The processor circuit 206 may be configured to execute computer readable instructions stored on the memory storage system of the control system 230. The processor circuit 206 may additionally be configured to generate outputs based on any suitable computer readable instructions the circuit 206 may execute. For example, the processor circuit 206 may generate an output configured to be received by a data acquisition device, such as the data acquisition device 224, to begin to receive data. Similarly, the processor circuit 206 may generate an output to be received by a blood flow alteration device, instructing the blood flow alteration device to begin to alter blood flow. In some embodiments, the processor circuit 206 may be further configured to process data received from the devices with which the control system 230 is in communication. In some embodiments, the processor circuit 206 may be configured to generate one or more graphical user interfaces to be output to a display, such as the display 208. In some embodiments, the processor circuit 206 may be additionally configured to receive user inputs from a user input device, such as the user input device 204.

The display 208 may be any suitable display. The display 208 may also be any suitable device. For example, the display 208 may include one or more pixels configured to display regions of an image to a user of the system 200. The display 208 may be in communication with the processor circuit 206 of the control system 230. In this way, the display 208 may receive instructions and/or images to display to a user of the system 200. In some embodiments, the display 208 may show a user a view of the data received and/or processed by the processor circuit 206. The display 208 may additionally convey various recommended actions or prompts for the user of the system 200 from the processor circuit 206. In some embodiments, the display 208 may additionally or alternatively be a user input device. For example, the user of the system 200 may select various elements within a graphic shown on the display 208 to direct the processor circuit 206 of the control system 230 to perform various actions or commands.

The data acquisition subsystem 201 may be in communication with the processor circuit 206, as shown in FIG. 2. The data acquisition subsystem 201 may be any suitable device, system, or subsystem. For example, the data acquisition subsystem 201 may be configured to receive commands from the processor circuit 206 of the control system 230 and send these commands or signals to one or more devices, such as the endovascular device 202. In some embodiments, the data acquisition subsystem 201 may process signals received from the processor circuit 206. In this way, the data acquisition subsystem 201 may facilitate communication between the processor circuit 206 and a device, such as the endovascular device 202. In some embodiments, the data acquisition subsystem 201 may be configured to control a data acquisition device 224. In this way, the data acquisition subsystem 201 and the data acquisition device 224 may together form a data acquisition device system. The data acquisition subsystem 201 may be configured to pre-process the data received from the data acquisition device 224. For example, the data acquisition subsystem 201 may smooth, average, or perform any other suitable preprocessing functions on the data received. The data acquisition subsystem 201 may then be configured to transmit the data received by the data acquisition device 224, which optionally may be preprocessed by the subsystem 201, to the processor circuit 206.

The carotid bodies stimulation subsystem 251 may be configured to control one or more stimulation devices. For example, the stimulation device may be the device 202. In some embodiments, the device 202 may include elements of a device configured to stimulate the carotid bodies of a patient. For example, the device 202 may include one or more electrodes configured to be positioned near the carotid bodies within the neck of a patient, near a juncture of the carotid artery. In some embodiments, and as shown in FIG. 2 the device 202 may include both a data acquisition device 224 and a stimulation device 254. In this way, the device 202 may be configured to both receive data and stimulate carotid bodies. The carotid bodies stimulation subsystem 251 may be configured to receive command signals from the processor circuit 206. For example, in response to a user input from the user of the system 200, or in response to other computer readable instructions, the processor circuit 206 may generate a command for the blood flow alteration carotid bodies stimulation subsystem 251 to begin to emit energy. In such an embodiment, the carotid bodies stimulation subsystem 251 may receive such a command from the processor circuit 206 and may generate one or more electrical pulses or electrical signals and transmit these pulses or signals to the device 254. Similarly, the processor circuit 206 may transmit a command to the carotid bodies stimulation subsystem 251 to stop stimulating the carotid bodies.

As shown in FIG. 2, the device 202 may be a single device configured to perform multiple functions. However, as will be described in greater detail hereafter, in some embodiments, a data acquisition device, such as the data acquisition device 224 may be housed on a separate device from the device containing the blood flow alteration device. In some embodiments, the device 202 may be an endovascular device or an external device. In some embodiments, both the data acquisition device 224 and stimulation device 254 may be positioned on the same endovascular device or separate endovascular devices. In other embodiments the data acquisition device 224 may be positioned on an endovascular device while the stimulation device is positioned on an external device or vice versa. In some embodiments, the data acquisition device 224 and the stimulation device 254 are positioned on the same external device or separate external devices.

As shown in FIG. 2, the data acquisition subsystem 201 and the carotid bodies stimulation subsystem 251 may be separate subsystems. In some embodiments, the data acquisition subsystem 201 may be in communication with the data acquisition device 224 of the device 202. Similarly, the carotid bodies stimulation subsystem 251 may be in communication with the stimulation device 254 of the same device 202. However, in some embodiments, the data acquisition subsystem 201 and the carotid bodies stimulation subsystem 251 may be the same subsystem. For example, this combined subsystem may be configured to both send and receive data or commands related to the acquisition of data and additionally send and receive commands related to the stimulation device 254.

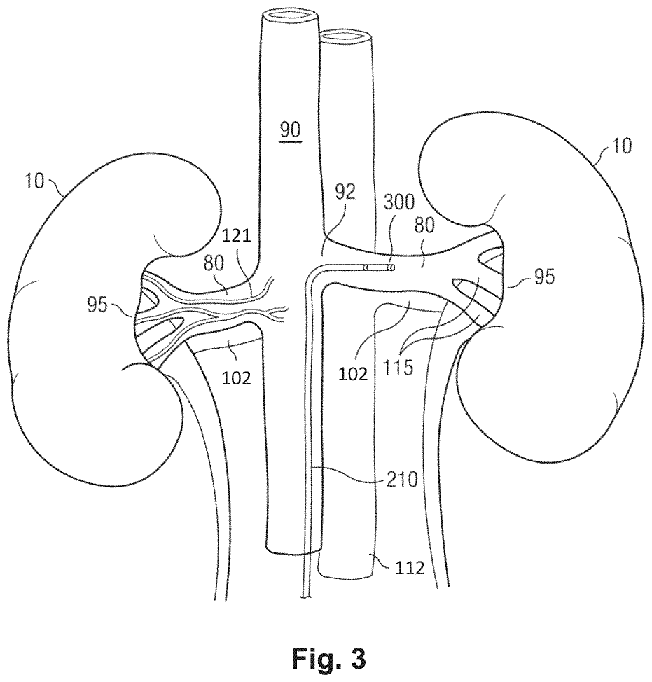

FIG. 3 illustrates an intravascular device 210 disposed within the human renal anatomy. The human renal anatomy includes kidneys 10 that are supplied with oxygenated blood by right and left renal arteries 80, which branch off an abdominal aorta 90 at the renal ostia 92 to enter the hilum 95 of the kidney 10. The abdominal aorta 90 connects the renal arteries 80 to the heart (not shown). Deoxygenated blood flows from the kidneys 10 to the heart via renal veins 102 and an inferior vena cava 112. Specifically, a flexible elongate member of the intravascular device 210 is shown extending through the abdominal aorta and into the left renal artery 80. In alternate embodiments, the intravascular device 210 may be sized and configured to travel through the inferior renal vessels 115 as well. Specifically, the intravascular device 210 is shown extending through the abdominal aorta and into the left renal artery 80. In alternate embodiments, the catheter may be sized and configured to travel through the inferior renal vessels 115 as well.

Left and right renal plexi or nerves 121 surround the left and right renal arteries 80, respectively. Anatomically, the renal nerve 121 forms one or more plexi within the adventitial tissue surrounding the renal artery 80. For the purpose of this disclosure, the renal nerve is defined as any individual nerve or plexus of nerves and ganglia that conducts a nerve signal to and/or from the kidney 10 and is anatomically located on the surface of the renal artery 80, parts of the abdominal aorta 90 where the renal artery 80 branches off the aorta 90, and/or on inferior branches of the renal artery 80. Nerve fibers contributing to the plexi arise from the celiac ganglion, the lowest splanchnic nerve, the corticorenal ganglion, and the aortic plexus. The renal nerves 121 extend in intimate association with the respective renal arteries into the substance of the respective kidneys 10. The nerves are distributed with branches of the renal artery to vessels of the kidney 10, the glomeruli, and the tubules. Each renal nerve 121 generally enters each respective kidney 10 in the area of the hilum 95 of the kidney, but may enter the kidney 10 in any location, including the location where the renal artery 80, or a branch of the renal artery 80, enters the kidney 10.

Proper renal function is essential to maintenance of cardiovascular homeostasis so as to avoid hypertensive conditions. Excretion of sodium is key to maintaining appropriate extracellular fluid volume and blood volume, and ultimately controlling the effects of these volumes on arterial pressure. Under steady-state conditions, arterial pressure rises to that pressure level which results in a balance between urinary output and water and sodium intake. If abnormal kidney function causes excessive renal sodium and water retention, as occurs with sympathetic overstimulation of the kidneys through the renal nerves 121, arterial pressure will increase to a level to maintain sodium output equal to intake. In hypertensive patients, the balance between sodium intake and output is achieved at the expense of an elevated arterial pressure in part as a result of the sympathetic stimulation of the kidneys through the renal nerves 121. Renal denervation may help alleviate the symptoms and sequelae of hypertension by blocking or suppressing the efferent and afferent sympathetic activity of the kidneys 10.

In some embodiments, the vessel 80 is a renal vessel and various metrics, such as pulse wave velocity, blood pressure, blood flow, fluid resistance, or any other metrics are determined in the renal artery. The processing system 230 may determine various physiological parameters, such as the blood pressure, blood flow, blood flow velocity, pulse wave velocity (PWV), strain or constriction of the vessel, voltage measurements of renal nerves, or any other parameters in the renal artery. The processing system 230 may determine a renal denervation therapy recommendation based on these parameters in a renal artery. For example, patients that are more likely or less likely to benefit therapeutically from renal denervation may be selected based on the parameters measured. In that regard, based on these parameters measured corresponding to the renal vessel, the processing system 230 can perform patient stratification for renal denervation.

FIG. 4 is a diagrammatic view of an endovascular device 402 within a renal anatomy, according to aspects of the present disclosure. The endovascular device 402 may be one embodiment of the device 202 described with reference to FIG. 2. FIG. 4 may illustrate a catheter-based system to measure the fluid resistance of blood flow from the renal artery into the kidney of a patient. Changes in the fluid resistance within the renal artery may correspond in response to stimulation of the sympathetic nervous system of the patient may be used to identify patients or assess success for renal denervation therapies. As shown in FIG. 4, the device 402 may be configured to be positioned within a blood vessel 400 of a patient. For example, as shown in FIG. 4, a diagrammatic view of a blood vessel 400 is provided. The blood vessel 400 may be a renal artery of the patient.

The endovascular device 402 may include physiological sensors to monitor blood flow and/or blood pressure. For example, the endovascular device 402 shown in FIG. 4 includes a flexible elongate member 410, a proximal pressure sensor 412, a distal pressure sensor 414, and a blood flow sensor 416. In some aspects, the proximal pressure, distal pressure, and blood flow sensors may acquire data which may be used to determine one or more blood fluid resistance values.

The flexible elongate member 410 may be sized and shaped, structurally arranged, and/or otherwise configured to be positioned within a artery 400 of a patient. The flexible elongate member 410 may be a part of guidewire and/or a catheter (e.g., an inner member and/or an outer member). The flexible elongate member 410 may be constructed of any suitable flexible material. For example, the flexible elongate member 410 may be constructed of a polymer material including polyethylene, polypropylene, polystyrene, or other suitable materials that offer flexibility, resistance to corrosion, and lack of conductivity. In some embodiments, the flexible elongate member 410 may define a lumen for other components to pass through. The flexible elongate member 410 may be sufficiently flexible to successfully maneuver various turns or geometries within the vasculature of a patient. The flexible elongate member 410 may be of any suitable length or shape and may have any suitable characteristics or properties.

The proximal pressure sensor 412, the distal pressure sensor 414, and the distal flow sensor 416 may acquire data and send it to the processor of the system (e.g., the processor circuit 206 of FIG. 2). For example, the proximal pressure sensor 412 may be configured to continuously acquire pressure data at a location 490 along the vessel 400. The distal pressure sensor 414 may be configured to continuously acquire pressure data at a location 492 along the vessel 400. The distal flow sensor 416 may be configured to continuously acquire flow data at a location 494 along the vessel 400. In some embodiments, the flow sensor 416 may obtain flow data corresponding to a volume of blood which passes through the location 494 of the vessel 400 over time. In other embodiments, the flow sensor 416 may obtain flow velocity data relating to the velocity of blood moving through the vessel. For example, the flow velocity data obtained by the flow sensor 416 may include a speed and position of blood cells along a cross-section area of the vessel 400 or a three-dimensional region of the vessel.

The processor circuit 206 may be configured to receive the pressure and flow data from the sensors of the device 402 to determine a fluid resistance measurement of the blood flow. A fluid resistance metric may correspond to the resistance of blood to flow through a particular length of the patient vasculature. In the embodiment shown in FIG. 4, the device 402 may calculate a blood flow resistance value corresponding to the length 480 of the vessel. The length 480 may correspond to a distance measurement between the proximal pressure sensor 412 and the distal pressure sensor 414. In some embodiments, a relationship between the pressure and flow data may be established by the processor 206 to determine the fluid resistance of the blood flow along the length 480. In some embodiments, fluid resistance along the length 480 may be described by the equation, F=ΔP/Q, where F is the fluid resistance, ΔP corresponds to a difference in pressure as measured by the distal pressure sensor 414 and the proximal pressure sensor 412, and Q corresponds to a flow measurement as measured by the flow sensor 416. It is understood that various constants or other variables may additionally affect the fluid resistance calculation as determined by the processor circuit 206 in response to various computer readable instructions stored on a memory in communication with the processor circuit.

The device 402 may be configured to measure fluid resistance as a metric to assess the sympathetic response to a stimulation of the carotid bodies. Alternatively, the processor circuit 206 may analyze other physiological measurements obtained by the device 402 to assess sympathetic response. For example, the processor circuit 206 may be configured to analyze a pressure measurement of the proximal pressure sensor 412 and/or the distal pressure sensor 414 to assess sympathetic response. Flow measurements from the flow sensor 416 may also be used to assess sympathetic response.

As shown in FIG. 4, a carotid artery 460 is shown within a neck of a patient. The carotid artery 460 and the renal artery 400 shown in FIG. 4 are of the same patient. As shown, the carotid artery 460 may be positioned within a location 450 of the neck of the patient. At a location along the carotid artery 460, one or more carotid bodies may be present. In some patients, the carotid body is a 2 to 6 mm, round bilateral sensory organ in the peripheral nervous system located in the adventitia of the bifurcation of the common carotid artery (e.g., the carotid artery 460). The carotid body may induce a physiologic response following changes in the vasculature by signaling the rest of the peripheral nervous system. Carotid bodies also have an interdependent regulatory relationship with other regulatory organs, such as the kidney. Stimulating the carotid body may include causing the carotid body to induce a physiologic response. This physiologic response may be a response of the sympathetic nervous system (e.g., a sympathetic response). In some patients, the carotid body may be stimulated, thus causing a sympathetic response, by applying physical pressure to the outside of the patient's neck at the location of the carotid body. This external pressure may be shown by the arrow 462. External pressure shown by the arrow 462 may be created in any suitable way. For example, a device may be positioned external of the patient in contact with the neck of the patient at the location of the carotid body. The device may be configured to apply pressure to the carotid body. In some embodiments, a physician may apply pressure in another way, such as by pressing on the neck of the patient at the location of the carotid body. As the carotid body receives this external pressure, it may induce a sympathetic response. This sympathetic response may include a drop in blood pressure within the vasculature, including at the renal artery 400, at the carotid artery 460, or at other locations. In some embodiments, this sympathetic response may include a drop in blood flow, or a change in flow resistance at any of these locations. As will be described in more detail hereafter, this sympathetic response may also include a change in the constriction of vessels in the patient, a change in the voltage corresponding to neuron impulses of nerves, such as the renal nerves, or other measured changes in the patient anatomy.

In one embodiment, the sympathetic response to pressure applied to the carotid bodies at the location 450 may be measured by the device 402. For example, as pressure is applied to the location 450 of the carotid body, one of the pressure sensors 412 or 414 of the device 402 may detect a change in the blood pressure, such as a drop in blood pressure. Similarly, the flow sensor 416 may detect a change in the blood flow, such as a drop in blood flow. As described previously, the device 402 may also acquire data used to calculate the fluid resistance along the length 480. The device 402 may, therefore, measure a change in the fluid resistance along the length 480, such as a drop or increase in the fluid resistance. Additional methods of measuring the anatomical response to pressure applied to the carotid body will be described hereafter.

Any of these changes in hemodynamic parameters may assist the physician. For example, if a change in any of these parameters (e.g., pressure, flow, fluid resistance, etc.) is observed in response to applied pressure to the carotid body, the physician, or a processor circuit of the system (e.g., the circuit 206), may determine that the patient is a good candidate for renal denervation. In other cases, after a renal denervation procedure has been performed, an observed change in any of these parameters may indicate that the renal denervation procedure was not successful. On the other hand, if these parameters do not change, indicating little to no response to the pressure applied to the carotid body, the physician or a processor circuit may determine that the patient is not a good candidate for renal denervation or that a renal denervation procedure was successfully performed.

Other methods of both stimulating the carotid body within a patient to induce a response as well as measuring the response will be disclosed hereafter. In some embodiments, the flow sensor 416 may be a thermoelectric sensor.

FIG. 5 is a diagrammatic view of the endovascular device 402 within a renal artery and an endovascular device 502 within a carotid artery, according to aspects of the present disclosure. According to some aspects of the present disclosure, FIG. 5 may illustrate a different way of stimulating the carotid body.

As shown in FIG. 5, an expanded view 500 of the location 450 is shown. The location of the expanded view 500 is shown by the indicator 501. As shown in the expanded view 500, an intravascular device 502 may be positioned within the carotid artery.

At a juncture of the carotid artery 560, a carotid body 562 is shown. The carotid body 562 may be stimulated by the device 502. In some embodiments, the device 502 may include a flexible elongate member 510, a pressure sensor 512, and a stimulation assembly 552. In some embodiments, the nerve stimulation assembly 552 may include multiple electrodes 554 placed on a corresponding number of arms. The arms of the stimulation assembly 552 may be configured to move the electrodes 554 in a radial direction. For example, as shown by the arrows 592 and 594, the electrodes 540 may move from a collapsed state to an expanded state. In an expanded state, the electrodes 554 may be moved in the direction radially outward shown by the arrows 594 so as to contact or come into close proximity with the walls of the carotid artery 560. In a collapsed state, the electrodes 554 may be moved in a direction radially inward shown by the arrows 592. In a collapsed state, the device 502 may move through the vasculature of a patient with greater ease.

In some embodiments, the device 502 may be configured for communication with a subsystem of the system 200. For example, the device 502 may be configured for communication with a nerve stimulation subsystem. The stimulation subsystem may send commands and/or signals to the device 502 causing the device 502 to move the electrodes 554 between an expanded and a collapsed state and/or to emit electrical pulses stimulating nerves such as the carotid body 562. In some embodiments, the carotid body 562 may be stimulated by the emission of electrical energy from the device 502. The stimulation of the carotid body 562 may cause any of the hemodynamic parameters previously described with reference to FIG. 4 to change. For example, the blood pressure of the patient, blood flow within the vasculature of the patient, or the fluid resistance of the blood at various locations, may be altered in response to the stimulation of the carotid body 562. In one example, the device 402 shown in FIG. 5 may be positioned within the renal artery 400. The system 200 may be configured to monitor a response to the stimulation of the carotid body 562 with the device 402 as described with reference to FIG. 4. In other embodiments, the system 200 may be configured to monitor the physiological response to the stimulation of the carotid body 562 with the device 502. For example, the device 502 may measure a change in the blood pressure of the patient with the device with the blood pressure sensor 512.

In some embodiments, the electrodes 554 of the device 502 may not be placed on expanding and contracting arms (e.g., the device 502 may not have expanding and contracting arms). For example, the electrodes 554 may alternatively be positioned on the flexible elongate member 510. In such instances, the electrodes 554 may be spaced from the inner wall of the vessel and may not contact the inner wall of the vessel. When excited, the electrodes 554 may emit electrical energy to stimulate the carotid bodies near the device 502 without direct contact.

In some embodiments, the blood pressure sensor 512 may be replaced with a flow sensor, or any other type of sensor. In such an embodiment, the device 502 may be configured to both stimulate the carotid body 562 and monitor physiological response to the stimulation. In such an embodiment, the device 402 may not be used. In this way, a single device (e.g., the device 502) may be the only endovascular device positioned within the vasculature of the patient.

An additional embodiment of the disclosure may include a stimulation device similar to the device 502 shown used in conjunction with an external patch (see, e.g., FIG. 6). In some aspects, the intravascular device 502 may include one or more monopolar electrodes. These electrodes may be in electrical communication with an electrical energy source. The external patch may be in communication with the same electrical energy source. In that regard, the monopolar electrodes of the intravascular device may serve as one (e.g., a live) of an electrical pair with the external patch, with the external patch serving as a ground electrode to complete a circuit. In that sense, when the intravascular electrode and/or external patch are activated, electrical energy may flow between the intravascular electrode and the external patch and thus stimulate one or more carotid bodies within the patient.

FIG. 6 is a diagrammatic view of the endovascular device 402 positioned within a renal anatomy and a carotid artery of a patient anatomy, according to aspects of the present disclosure. FIG. 6 may illustrate a different way of stimulating the carotid body. FIG. 6 illustrates an external patch configured to stimulate the carotid body of a patient. It is noted that the patch described with reference to FIG. 6, as well as any other patch or external stimulation or measurement device described or illustrated herein may refer to a patch or any other device. For example, an external stimulation or measurement device, such as the device 660 shown in FIG. 6, may include a pad or cuff that extends around a neck (or in other embodiments, any limb or other anatomical part) of a patient. For example, the device 660 may be a part of a device which is affixed to a patient by a band or cuff which extends around the neck. In this way, the band or cuff may press the patch or pad against the neck of the patient such that it is tightly positioned against the skin of the patient. The band or cuff may ensure that the patch or pad remains positioned next to the carotid artery and/or carotid bodies of the patient.

As shown in FIG. 6, the device 402 may be positioned within the renal artery 400 of the patient. This device 402 may be configured to monitor a sympathetic response the stimulation of the carotid body of the patient. Also shown in FIG. 6, is the device 660. The device 660 may include an external patch 660 configured to be affixed to the outer surface of the patient's skin at the location of the carotid body. In some embodiments, the patch 660 may include one or more electrodes in communication with a nerve stimulation subsystem of the system 200. In some embodiments, the patch 660 may be configured to emit electrical energy which in turn may stimulate the carotid body of the patient. In response to the stimulation of the carotid body, any of the physiological parameters previously described may be changed. This change in physiological parameters may be monitored by the device 402.

FIG. 7 is a diagrammatic view of an endovascular device 702 positioned within a renal anatomy, according to aspects of the present disclosure. FIG. 7 shows a device which may be used to monitor the sympathetic response of the patient in response to stimulation of the carotid bodies. The device 702 may be the device 202 described with reference to FIG. 2. As shown in FIG. 7, the device 702 may be configured to be positioned within the renal artery 400 of a patient. In other embodiments, the device 702 may be positioned within the carotid artery of a patient, or in any other blood vessel of the patient. In some aspects, the stimulation device and the anatomical measurement device may be the same device.

As shown, the device 702 may include a structure 720. In the embodiment shown in FIG. 7, multiple electrodes 722 may be positioned on an outer surface of the structure 720. In that regard, the electrodes 722 may be configured to obtain voltage measurements. The electrodes 722 may be a part of a data acquisition device (e.g., the device 224 of FIG. 2). The structure 720 may be similar to the stimulation assembly 552 described with reference to FIG. 5. For example, the structure 720 may be configured to move the electrodes 722 in a radially outward and inward direction. For example, with the device 702 positioned within a renal artery, as the structure 720 is expanded, the electrodes 722 may detect neural impulses sent from and/or to the central nervous system. The electrodes 722 may identify a response of the sympathetic nervous system by detecting changes in potential or voltage within the renal nerves, corresponding to a neural impulse being sent or received. This data may be used to identify whether a patient is likely to respond positively to a renal denervation procedure, whether a particular side branch or other location is a good location for renal denervation, or whether a renal denervation procedure was successful. Similarly, the device 702 may monitor neural impulses of nerves within any other vessel of the patient.

As an example, if a change in voltage is observed in response to a stimulation of the carotid bodies, the user of the system 200 or a processor circuit may determine that the patient is a good candidate for renal denervation. Alternatively, if a change is observed after a renal denervation procedure, the renal denervation procedure may have been unsuccessful. However, if little to no change in voltage is detected by the electrodes 722, the patient may not be a good candidate for renal denervation, or if after a renal denervation procedure, the procedure may have been successful.

Aspects of the structure 720, the assembly 552 (FIG. 5), and/or the structure 820 (FIG. 8) may include features described in U.S. patent application Ser. No. 13/458,856 (Atty. Docket No. 2012P02290US/44755.805US01), titled, “METHODS AND APPARATUS FOR RENAL NEUROMODULATION” and filed Apr. 27, 2012, which is hereby incorporated by reference in its entirety.

In some aspects, the structure 720 may be or include a compliant balloon. For example, the structure 720 may be a balloon which may be inflated within the renal artery 400. As the structure 720 is inflated, the blood flow through the renal artery 400 may be restricted. As the balloon is dilated and blood flow is restricted, the sympathetic system may be stimulated. Full dilation of the balloon will put the balloon surface in contact with the intimal surface of the renal artery, fully restricting blood flow. The reduction of flow to the kidney and reduction of pressure will alter the sympathetic drive from the renal nerve. This in turn will impact the patient's blood pressure and/or fluid resistance of blood within the renal artery. Blood pressure/fluid resistance changes over time indicate the patient's receptiveness to renal denervation therapies. The sympathetic nervous system response may be monitored by electrodes 722 or by any other method described herein.

FIG. 8 is a diagrammatic view of an endovascular device 802 positioned within a renal anatomy, according to aspects of the present disclosure. FIG. 8 shows another device which may be used to monitor the sympathetic response of the patient in response to stimulation of the carotid bodies or any other stimulation of the sympathetic nervous system. FIG. 8 is a schematic diagram of an endovascular device 802, according to aspects of the present disclosure. The device 802 may be the device 202 described with reference to FIG. 2. As shown in FIG. 8, the device 802 may be configured to be positioned within the renal artery 400 of a patient. In other embodiments, the device 802 may be positioned within the carotid artery of a patient, or in any other blood vessel of the patient.

As shown, the device 802 may include a structure 820. In the embodiment shown in FIG. 8, a strain sensor 822 may be positioned on the structure 820. The strain sensor 822 may be a part of a data acquisition device (e.g., the device 224 of FIG. 2). The structure 820 may be similar to the stimulation assembly 552 described with reference to FIG. 5. For example, the structure 820 may be configured to move the strain sensor 822 in a radially outward and inward direction. For example, with the device 802 positioned within a renal artery, after the structure 820 is expanded, the strain sensor may detect changes in the tone of the vessel 400 (e.g., an increase or decrease in pressure applied by the vessel wall on the strain sensor 822 or the extent to which the vessel 400 contracts or expands). In that regard, the strain sensor 822 may be configured to obtain strain measurements. This data may be used to identify whether a patient is likely to respond positively to a renal denervation procedure, whether a particular side branch or other location is a good location for renal denervation, or whether a renal denervation procedure was successful. Similarly, the device 802 may monitor strain of a vessel within any other vessel of the patient.

As an example, if a change in vessel contraction is observed in response to stimulation of the carotid bodies, the user of the system 200 or a processor circuit may determine that the patient is a good candidate for renal denervation. Alternatively, if a change is observed after a renal denervation procedure, the renal denervation procedure may have been unsuccessful. However, if little to no change in contraction is detected by the strain sensor 822, the patient may not be a good candidate for renal denervation, or if after a renal denervation procedure, the procedure may have been successful.

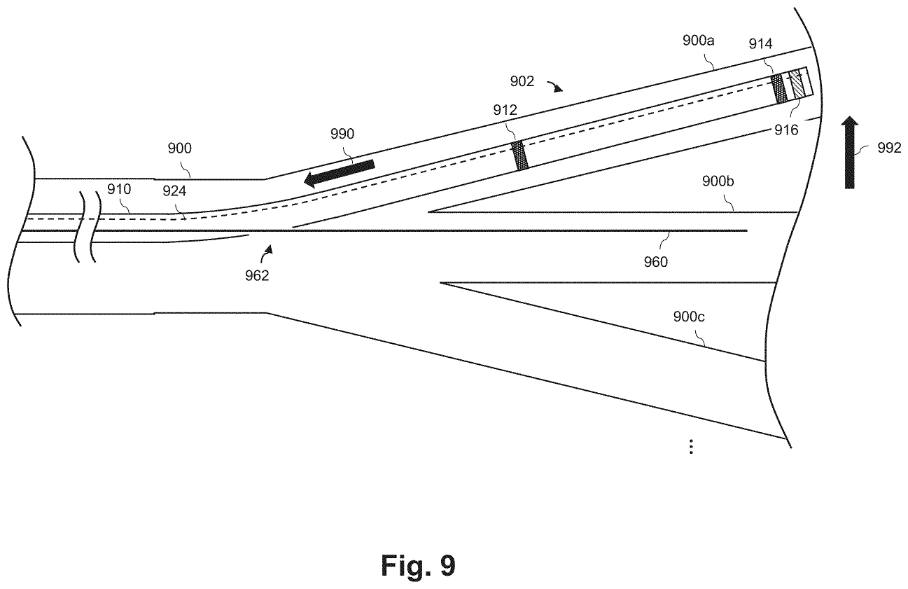

FIG. 9 is a schematic diagram of an endovascular device 902, according to aspects of the present disclosure. The device 902 may be the device 202 described with reference to FIG. 2. As shown in FIG. 9, the device 902 may be configured to be positioned within a blood vessel of a patient. The device 902 shown in FIG. 9, like the devices previously described, may include structures configured to monitor the sympathetic response to the reduction in blood flow.

A renal artery 900 is shown in FIG. 9. The renal artery 900 may, at a distal end, split into multiple side branches. For example, a side branch 900a, a side branch 900b, and a side branch 900c are shown. It is noted that additional or fewer side branches may be included within the renal vasculature.

In the embodiment shown, a portion of the device 902 may be positioned within one side branch (e.g., the side branch 900a) while a separate portion of the device 902 may be positioned within a different side branch (e.g., the side branch 900b). In some embodiments, the measurement portion of the device 902 (e.g., a proximal pressure sensor 912, a distal pressure sensor 914, and/or a distal flow sensor 916) may be moved to different side branches within the renal vasculature without completely removing the device 902.

As shown in FIG. 9, a guidewire 960 may extend along the longitudinal center of the device 902. In some embodiments, the guidewire 960 may be positioned within the renal artery first. In the embodiment shown, the guidewire 960 may be positioned within the side branch 900b. The device 902 may then be positioned around the guidewire 960. For example, a lumen of the device 902 may be sized to receive the guidewire 960. At the opening 962, the device 902 may be positioned around the guidewire 960. The device 902 may then move along the guidewire through the patient vasculature to the renal vasculature. There, the device 902 may be positioned within the same side branch 900b with the guidewire 960. After measurements are made there, however, the device 902 may be moved in a proximal direction so as to exit the side branch 900b and return to the primary renal artery 900. There, the measurement portion of the device 902 may be deflected from the guidewire 960 so as to be positioned in a separate side branch (e.g., the side branch 900a) while the guidewire 960 remains in the same side branch (e.g., the side branch 900b).

In some embodiments, the device may include one or more pull wires 914. A pull wire (e.g., the pull wire 924) may be positioned within the device 902 or on an outer surface of the device 902. In some embodiments, the pull wire 924 may be attached to a side of the device 902 or a side of the flexible elongate member 910 of the device 902. In this way, when a physician, or other automated or robotic system, pulls on the pull wire 924, a force is exerted in the proximal direction shown by the arrow 990. Due to the flexible nature of the device 902, this force on one side of the device 902 causes the device to deflect away from the guidewire 960 in a direction corresponding the to the location at which the pull wire 924 is attached to the device. This direction may be shown by the arrow 992.

FIG. 10 is a diagrammatic view of an external device 1010, according to aspects of the present disclosure. FIG. 10 illustrates the device 1010 positioned around the neck of a patient. In some embodiments, the device 1010 may be positioned around the neck of a patient such that it overlaps a portion corresponding to the carotid artery. For example, the location 450 of the carotid artery is shown in FIG. 10. In some aspects, the external device 1010 may be a fluid filled tube or a wrap such as a flexible wrap which may be wrapped around the neck of a patient.

An expanded view 1052 of the device 1010 is shown in FIG. 10. The location of the expanded view 1052 may be identified by the indicator 1050. As shown in the expanded view 1052, the device 1010 may include a strain sensor 1022. In addition, the device 1010 may be a fluid filled structure. For example, a fluid 1040 may be positioned within a central lumen of the device 1010. In some embodiments, the device 1010 may include a flexible outer membrane configure to contain the fluid 1040. In some embodiments, the device 1010 may include more than one strain sensor 1022. For example, multiple strain sensors 1022 may be positioned along the device 1010.

In some embodiments, the strain sensor 1022 may be similar to the strain sensor 822 described with reference to FIG. 8. For example, the strain sensor 1022 may be configured to measure the pressure or movement of a surface with which the strain sensor 1022 is in contact. In some embodiments, the strain sensor 1022 may monitor a pressure exerted on the strain sensor 1022. In this way, the strain sensor 1022 of the device 1010 may monitor the tone of the outer skin of the patient. For example, the strain sensor 1022 may monitor whether the muscles of the neck of the patient, or the vasculature within the neck of the patient, expands, contracts, tightens, or loosens, or is strained or tense or exhibits any other changes in features or characteristics. In some embodiments, any of these physiological parameters may be measured by the strain sensor 1022 may correspond to a sympathetic response of the patient. In this way, the device 1010 may be used to monitor the sympathetic response of the patient to stimulation of the carotid bodies.

FIG. 11 is a diagrammatic view of carotid arteries of a patient anatomy, according to aspects of the present disclosure. FIG. 11 illustrates an additional method of monitoring sympathetic response to carotid body stimulation in the patient. Specifically, FIG. 11 includes two regions corresponding to carotid arteries within a neck of the patient. For example, a region 1150 may correspond to the location of a left carotid artery of a patient. The region 1152 may correspond to a location of the right carotid artery of a patient.

As shown in FIG. 11, an external device 1162 may be affixed to the outer surface of the skin of the neck of the patient. In some embodiments, the device 1162 may be a patch. The device 1162 may include one or more electrodes. the electrodes if the device 1162 may be configured to monitor the sympathetic response of the patient. For example, the electrodes at the device 1162 may be configured to monitor neural impulses of nerves associated with the vasculature of the patient. In this way, the device 1162 may monitor a sympathetic response to the stimulation of the carotid bodies.

As explained herein, any of the devices used to stimulate the carotid bodies of the patient may be used with any of the devices to monitor the sympathetic response. For example, an external device used to stimulate the carotid bodies of patient may be used in conjunction with an internal device, or an endovascular device, configured to monitor the sympathetic response of the patient. In some embodiments, an internal device configured to stimulate the carotid bodies maybe used it correspondence with an external device used to monitor the sympathetic response. Any combination of the devices disclosed herein is fully anticipated.

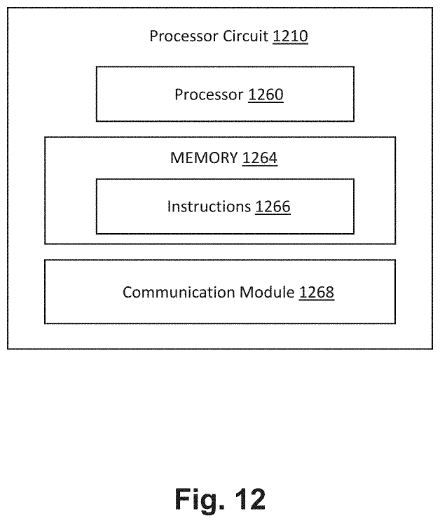

FIG. 12 is a schematic diagram of a processor circuit, according to aspects of the present disclosure. The processor circuit 1210 may be implemented in the control system 230 (e.g., as shown in FIG. 2), or any other suitable location. In an example, the processor circuit 1210 may be in communication with any of the devices, systems, or subsystems described in the present disclosure. For example, the processor circuit 1210 may be in communication with a blood flow sensing device, a pressure sensing device, an extraluminal imaging device, a nerve stimulation device, a nerve ablation device or any other device, system, or subsystem. The processor circuit 1210 may include a processor 106 and/or a communication interface. One or more processor circuits 1210 are configured to execute the operations described herein. As shown, the processor circuit 1210 may include a processor 1260, a memory 1264, and a communication module 1268. These elements may be in direct or indirect communication with each other, for example via one or more buses.

The processor 1260 may include a CPU, a GPU, a DSP, an application-specific integrated circuit (ASIC), a controller, an FPGA, another hardware device, a firmware device, or any combination thereof configured to perform the operations described herein. The processor 1260 may also be implemented as a combination of computing devices, e.g., a combination of a DSP and a microprocessor, a plurality of microprocessors, one or more microprocessors in conjunction with a DSP core, or any other such configuration.

The memory 1264 may include a cache memory (e.g., a cache memory of the processor 1260), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 1264 includes a non-transitory computer-readable medium. The memory 1264 may store instructions 1266. The instructions 1266 may include instructions that, when executed by the processor 1260, cause the processor 1260 to perform the operations described herein with reference to any of the devices, system, or subsystems described. Instructions 1266 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 1268 can include any electronic circuitry and/or logic circuitry to facilitate direct or indirect communication of data between the processor circuit 1210, the devices, systems, or subsystems described herein, the display 208, processor circuit 206, or user input device 204 (FIG. 2). In that regard, the communication module 1268 can be an input/output (I/O) device. In some instances, the communication module 1268 facilitates direct or indirect communication between various elements of the processor circuit 1210 and/or various described endovascular or extraluminal devices, systems, and/or the system 230 (FIG. 2).

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A system, comprising:
a processor circuit configured for communication with an anatomical measurement device, wherein the processor circuit is configured to:
receive, from the anatomical measurement device, a first metric associated with a first sympathetic response of the patient while a sympathetic nervous system of the patient is not under stimulation;
generate a visual representation of the first metric;
receive, from the anatomical measurement device, a second metric associated with a second sympathetic response of the patient while the sympathetic nervous system of the patient is under the stimulation, wherein the stimulation of the sympathetic nervous system comprises stimulation of a carotid body of the patient;
generate a visual representation of the second metric; and
output a screen display to a display in communication with the processor circuit, wherein the screen display comprises the visual representation of the first metric and the visual representation of the second metric.

2. The system of claim 1,
wherein the processor circuit is configured to:
perform a comparison of the first metric and the second metric; and
determine, based on the comparison, a likelihood of success of a future renal denervation procedure for the patient,
wherein the screen display comprises a visual representation based on the likelihood of success.

3. The system of claim 1,
wherein the processor circuit is configured to:
perform a comparison of the first metric and the second metric; and
determine, based on the comparison, a degree of success a completed renal denervation procedure for the patient,
wherein the screen display comprises a visual representation based on the degree of success.

4. The system of claim 1, wherein the anatomical measurement device comprises an endovascular catheter or guidewire configured to be positioned within a blood vessel of a patient.

5. The system of claim 4, wherein the blood vessel comprises a renal artery of the patient.

6. The system of claim 4,
wherein the endovascular catheter or guidewire comprises one or more pressure sensors and one or more flow sensors, and
wherein the processor circuit is configured to determine a fluid resistance measurement based on data received from the one or more pressure sensors and the one or more flow sensors.

7. The system of claim 4, wherein the endovascular catheter or guidewire comprises a strain sensor.

8. The system of claim 4, wherein the endovascular catheter or guidewire comprises one or more electrodes configured to measure an electrical field.

9. The system of claim 1, wherein the anatomical measurement device is configured to be positioned outside of the patient and in contact the patient's skin.

10. The system of claim 9, wherein the anatomical measurement device comprises a strain sensor.

11. The system of claim 9, wherein the anatomical measurement device includes one or more electrodes configured to measure an electrical field.

12. The system of claim 1,
wherein the processor circuit is configured for communication with a stimulation device, and
wherein the processor circuit is configured to control the stimulation device to provide the stimulation of the carotid body.

13. The system of claim 12, wherein the stimulation device comprising an endovascular catheter or guidewire configured to be positioned within a carotid artery of the patient.

14. The system of claim 12, wherein the stimulation device is configured to be positioned outside of the patient.

15. The system of claim 12, wherein the stimulation device comprises one or more electrodes configured to provide the stimulation of the carotid body.

16. The system of claim 1, wherein the stimulation of the carotid body comprises application of external pressure to a neck of the patient at a region comprising the carotid body.

17. A method, comprising:
receiving, with a processor circuit, first metric associated with a first sympathetic response of the patient from an anatomical measurement device in communication with the processor circuit, wherein the first metric is obtained by the anatomical measurement device while a sympathetic nervous system of the patient is not under stimulation;
generating, with the processor circuit, a visual representation of the first metric;
receiving, with the processor circuit, a second metric associated with a second sympathetic response of the patient from the anatomical measurement device, wherein the second metric is obtained by the anatomical measurement device while the sympathetic nervous system of the patient is under stimulation, wherein the stimulation of the sympathetic nervous system comprises stimulation of a carotid body of the patient;
generating, with the processor circuit, a visual representation of the second metric; and
outputting, with the processor circuit, a screen display to a display in communication with the processor circuit, wherein the screen display comprises the visual representation of the first metric and the visual representation of the second metric.

18. A system, comprising:
an anatomical measurement device; and
a processor circuit configured for communication with the anatomical measurement device and a display, wherein the processor circuit is configured to:
receive, from the anatomical measurement device, a first metric associated with a first sympathetic nervous system response of a patient while a carotid body of the patient is not under stimulation;
receive, from the anatomical measurement device, a second metric associated with a second sympathetic nervous system response of a patient while the carotid body is under the stimulation, the stimulation of the carotid body causing a change from the first sympathetic nervous system response to the second sympathetic nervous system response;
generate a screen display comprising a visual representation of the first metric and a visual representation of the second metric; and
output the screen display to the display.
